(19) United States
(12) Patent Application Publication
(10) Pub. No.: US 2020/0077636 A1
(43) Pub. Date: Mar. 12, 2020

(54) LIQUID DIFFUSION DEVICE

(71) Applicant: HSUAN WANG, Taipei City (TW)

(72) Inventor: HSUAN WANG, Taipei City (TW)

(21) Appl. No.: 16/127,239

(22) Filed: Sep. 11, 2018

(51) Int. Cl. A01M 1/20; A61L 9/12; F23D 3/40

(52) U.S. Cl. CPC A01M 1/2044; B32B 3/26; F23D 3/40; A61L 9/12

(57) ABSTRACT

A liquid diffusion device, comprising: a liquid storage container for receiving liquid; a core capable of being putted into the liquid storage container for sucking the liquid within the liquid storage container; and a diffusion sheet being formed by a porous material, and installed at an upper side of the liquid storage container to be in contact with the core; thereby, the core can suck the liquid within the liquid storage container and then the liquid is transferred to the diffusion sheet continuously. The diffusion sheet is sintered glass with pores, or is formed by compressing and solidifying alloy powders with a plurality of pores for remaining therein. The outer container is installed with an upper cover which covers or screws on the outer container. The diffusion sheet has a regular shape or an irregular shape.

### FIELD OF THE INVENTION

The present invention is related to a liquid diffusion device, and in particular to a liquid diffusion device, in that, the diffusion sheet is sintered glass with pores, or is formed by compressing and solidifying alloy powders with a plurality of pores for remaining therein so that the diffusion sheet has the effect of diffusion of fragrance continuously or the effect of burning the fuel continuously.

### BACKGROUND OF THE INVENTION

In the prior art a device for fragrance diffusion of essential oil, an fragrance diffusion element is installed in a container so that fragrance of the liquid within the container can be diffused. Furthermore the device has a convex bottom so that the device can stand steadily without falling down and thus the liquid within the container will not fall out. The fragrance diffusion element is a wood with a plurality of capillaries for sucking the liquid to the wood surfaces.

However, the fragrance diffusion woods are not uniform as they are manufactured in different working process and batches. The capillaries are easy to be blocked so that the fragrance liquid cannot be sucked effectively. Furthermore due to heat expansion and cool contraction, the woods will become worse and thus to be destroyed.

### SUMMARY OF THE INVENTION

Accordingly, the object of the present invention is to provide a liquid diffusion device, wherein the glass sintered from glass powder or metal may be as material of the diffusion sheet 3 so that the diffusion sheet 3 has a preferred capillary effect and the quality thereof may be steady. Furthermore it can be manufactured easily. This kind of diffusion sheet 3 may be used repeatedly without the problem that the pores are blocked. Furthermore these kinds of material can prevent the effects of heat expansion and cool reduction. It provides a long time period and steady suction effect without longer lifetime. Thereby, the present invention can be used to different generations and has the advantages for storage.

To achieve above object, the present invention provides a liquid diffusion device, comprising: a liquid storage container for receiving liquid; a core capable of being putted into the liquid storage container for sucking the liquid within the liquid storage container; and a diffusion sheet being formed by a porous material, and installed at an upper side of the liquid storage container to be in contact with the core; thereby, the core can suck the liquid within the liquid storage container and then the liquid is transferred to the diffusion sheet continuously. The diffusion sheet is sintered glass with pores, or is formed by compressing and solidifying alloy powders with a plurality of pores for remaining therein. The liquid storage container is formed by an inner container and an outer container; and the inner container serves to store liquid; and the core is placed into the inner container; and the outer container serves to receive the inner container therein. The outer container is installed with an upper cover which covers or screws on the outer container. The diffusion sheet has a regular shape or an irregular shape.

### BRIEF DESCRIPTION OF THE DRAWINGS

FIG. 1 is an assembled perspective view of the preferred embodiment of the present invention.

FIG. 2 is an exploded perspective view of the preferred embodiment of the present invention.

FIG. 3 is an assembled lateral view of the preferred embodiment of the present invention.

FIG. 4 is a cross sectional view along line A-A of FIG. 3.

FIG. 5 is an exploded perspective view of a preferred embodiment of the present invention.

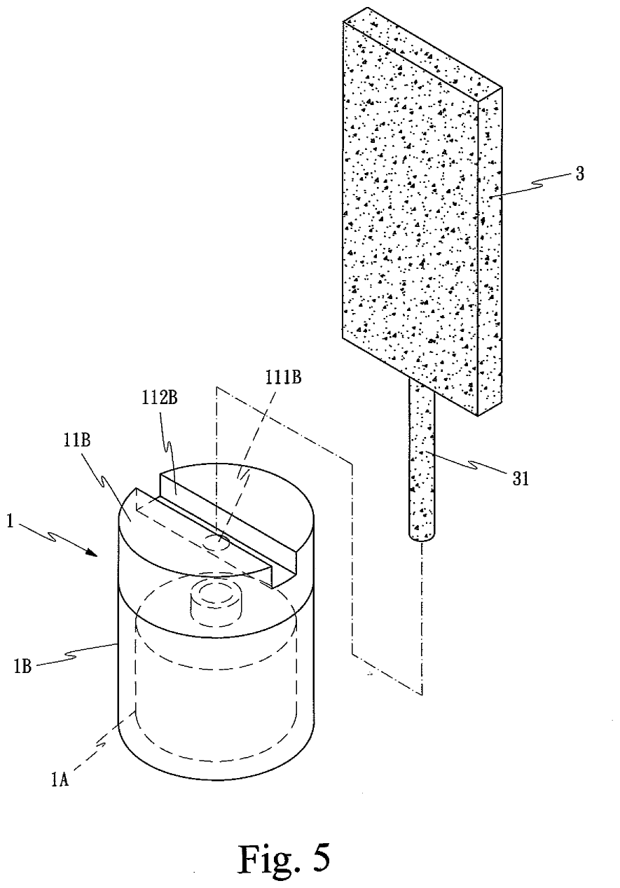

FIG. 6 is an assembled perspective view of a preferred embodiment of the present invention.

FIG. 7 is a schematic view showing the operation of the present invention, wherein the diffusion sheet serves to suck alcohol or other fuel to fire them continuously.

FIG. 8 is an assembled cross sectional view about another embodiment of the present invention.

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

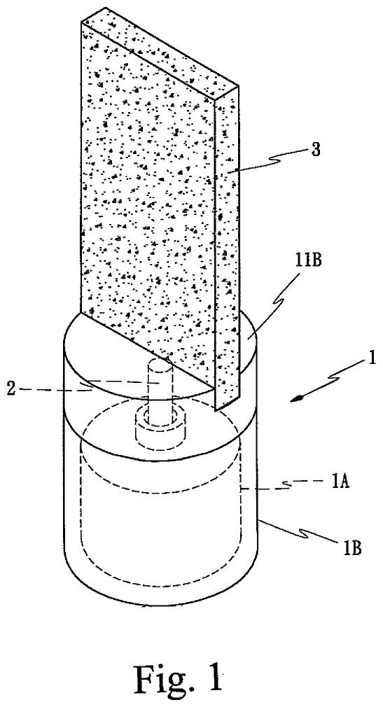

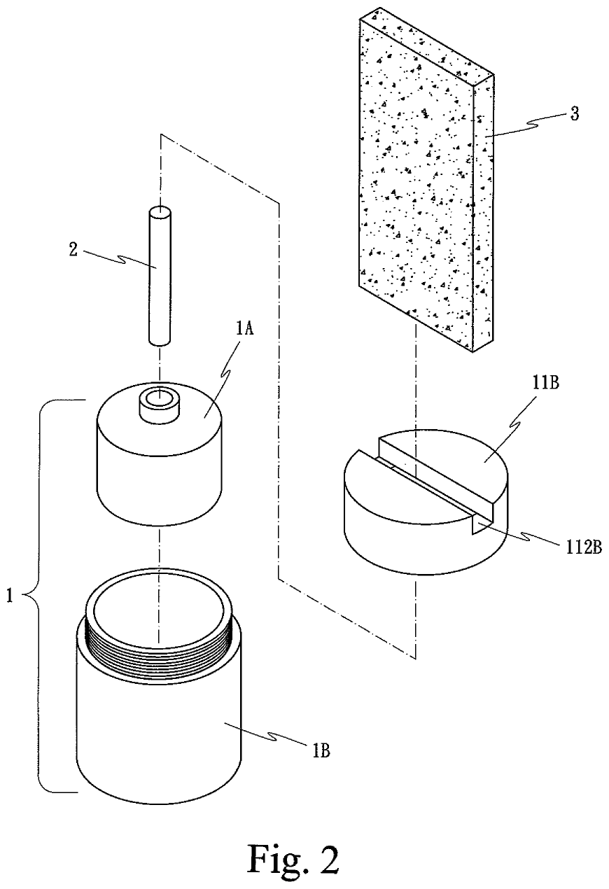

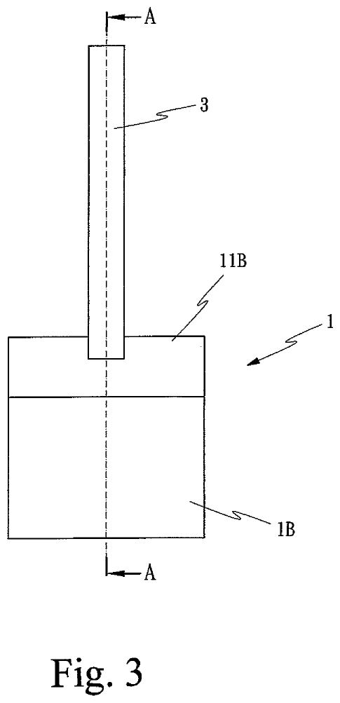

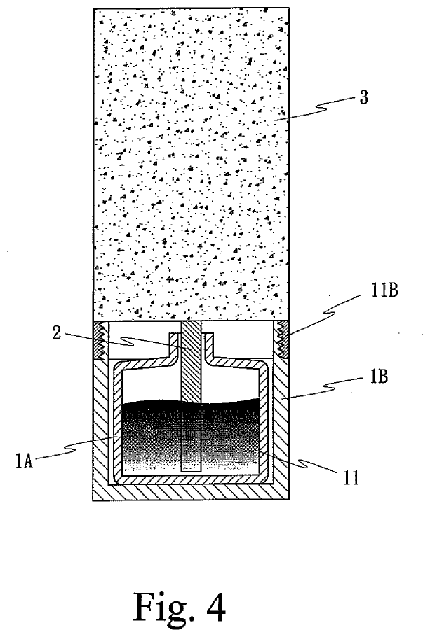

With reference to FIGS. 1 to 4, the liquid diffusion device of the present invention is illustrated. The present invention includes the following elements.

A liquid storage container 1 serves for receiving liquid 11, such as perfumes, essential oils, alcohol, fuels, etc.

A core 2 is capable of being putted into the liquid storage container 1 for sucking the liquid 11 within the liquid storage container 1.

A diffusion sheet 3 is a porous material (for example, manmade porous material), and is installed at an upper side of the liquid storage container 1 to be in contact with the core 2. Thereby, the core 2 can suck the liquid 11 within the liquid storage container 1 and then the liquid 11 is transferred to the diffusion sheet 3 continuously. Preferably, the diffusion sheet 3 is sintered glass with pores, or metal which is compressed and solidified from powders so that the diffusion sheet 3 may diffuse the liquid 11 quickly and the liquid 11 can be diffused continuously by diffusing (such as perfume) or to be burned (such as alcohol).

The liquid storage container 1 is formed by an inner container 1A and an outer container 1B. The inner container 1A serves to store liquid 11, such as perfumes, essential oils or alcohol, or fuels, etc. The core 2 is placed into the inner container 1A, The outer container 1B serves to receive the inner container 1A therein. Thereby, there may be a plurality of inner containers 1A which serves to store different kinds of liquids 11. The different kinds of inner containers 1A may be matched with different kinds of outer container 1B. Furthermore the outer container 1B is updated and the inner containers 1A are selectable for assembling as a set.

The outer container 1B may be installed with an upper cover 11B which covers or screws on the outer container 1B. The upper cover 11B may be formed with a penetrating hole 111B for passing through the core 2. Furthermore the upper side of the upper cover 11B may be formed with an embedding groove 112B so that a distal end of the diffusion sheet 3 may be embedded into the groove 112B and is positioned therein.

In the present invention, the glass sintered from glass powder or metal may be as material of the diffusion sheet 3 so that the diffusion sheet 3 has a preferred capillary effect and the quality thereof may be steady. Furthermore it can be manufactured easily. This kind of diffusion sheet 3 may be used repeatedly without the problem that the pores are blocked. Furthermore these kinds of material can prevent the effects of heat expansion and cool reduction. It provides a long time period and steady suction effect without longer lifetime. Thereby, the present invention can be used to different generations and has the advantages for storage.

Other than as an independent core, the core 2 may be formed with the diffusion sheet 3 and is extended from a lower side of the diffusion sheet 3 as an integral structure. At this case, the core 2 and the diffusion sheet 3 are formed with identical material. Furthermore the core 2 is directly in contact with the liquid 11 in the liquid storage container 1.

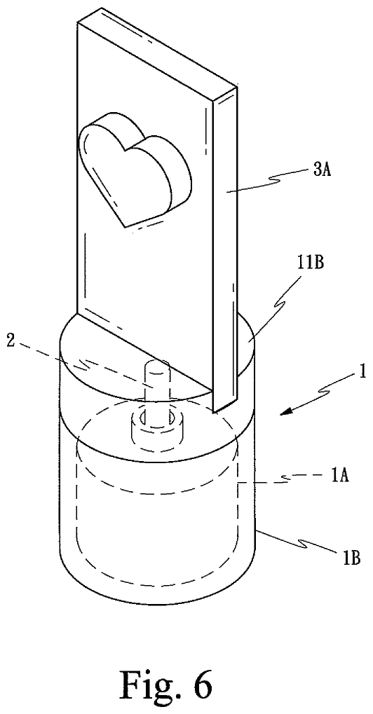

Furthermore the shape of the diffusion sheet 3 is not confined to have regular geometrical shape, other irregular shape is also permissible, such as shapes of animals, flowers, grasses, figures, people, textures, words, alphabets, other trademarks, or signs, drawings, etc. All these are within the scope of the present invention. FIG. 6 shows the diffusion sheet 3A with a specific shape.

As illustrated in above embodiments, the liquid storage container 1 may contain an inner container 1A and an outer container 1B. The inner container 1A serves to store liquid 11 and a core 2 is placed within the inner container 1A. The outer container 1B serves to receive the inner container 1A. Thereby, different inner containers 1A may be stored with different kinds of liquids 11 and thus is matched to a desired outer container 1B. However, matching of the inner container 1A and the outer container 1B is selectable and updatable.

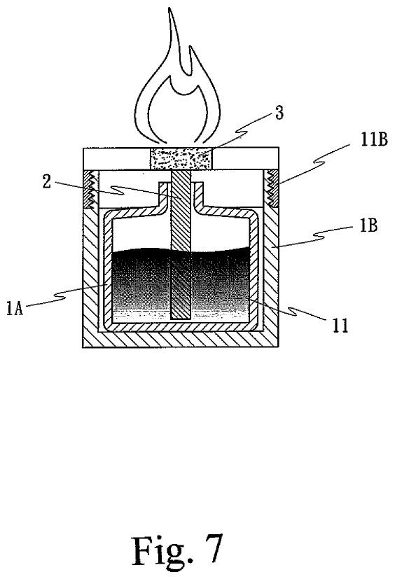

Furthermore in the present invention, when the liquid storage container 1 stores perfume**, the diffusion sheet 3 has the effect of diffusion of the perfume** and when the liquid storage container 1 stores alcohol or other fuel, the diffusion sheet 3 has the effect of firing continuously, referring to FIG. 7.

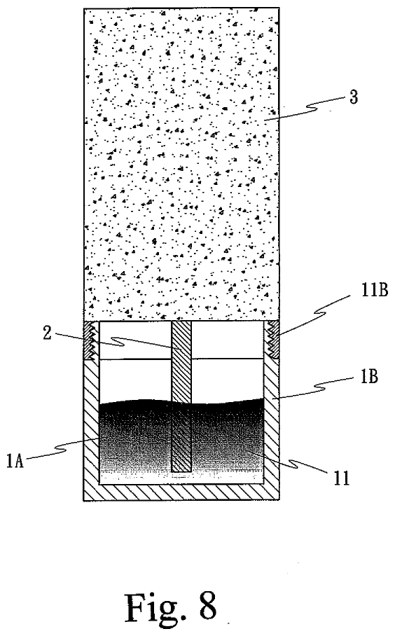

In the present invention, the liquid storage container 1 may be a single container, for example, the inner container 1A is neglected as illustrated in FIG. 8, or the outer container 1B is neglected. However, all these designs may achieve the above mentioned effect of the present invention.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A liquid diffusion device, comprising:
a liquid storage container for receiving liquid;
a core capable of being putted into the liquid storage container for sucking the liquid within the liquid storage container; and
a diffusion sheet being formed by a porous material, and installed at an upper side of the liquid storage container to be in contact with the core;
thereby, the core can suck the liquid within the liquid storage container and then the liquid is transferred to the diffusion sheet continuously.

2. The liquid diffusion device as claimed in claim 1, wherein the diffusion sheet is sintered glass with pores.

3. The liquid diffusion device as claimed in claim 1, wherein the diffusion sheet is formed by compressing and solidifying alloy powders with a plurality of pores for remaining therein.

4. The liquid diffusion device as claimed in claim 1, wherein the liquid storage container is formed by an inner container and an outer container; and the inner container serves to store liquid; and the core is placed into the inner container; and the outer container serves to receive the inner container therein.

5. The liquid diffusion device as claimed in claim 1, wherein the outer container is installed with an upper cover which covers or screws on the outer container.

6. The liquid diffusion device as claimed in claim 1, wherein the upper cover is formed with a penetrating hole for passing through the core.

7. The liquid diffusion device as claimed in claim 1, wherein an upper side of the upper cover is formed with an embedding groove so that a distal end of the diffusion sheet can be embedded into the groove and is positioned therein.

8. The liquid diffusion device as claimed in claim 1, wherein the core is integrally formed at lower end of the diffusion sheet and the core and the diffusion sheet are formed by a same material.

9. The liquid diffusion device as claimed in claim 1, wherein the diffusion sheet has a regular shape.

10. The liquid diffusion device as claimed in claim 1, wherein the diffusion sheet has an irregular shape.
